(19) United States
(12) Patent Application Publication
(10) Pub. No.: US 2024/0023832 A1
(43) Pub. Date: Jan. 25, 2024

(54) Application to detect dyslexia using Support Vector Machine and Discrete Fourier Transformation technique.

(71) Applicant: Rohan Jay, Pennington, NJ (US)

(72) Inventor: Rohan Jay, Pennington, NJ (US)

(21) Appl. No.: 18/479,087

(22) Filed: Oct. 1, 2023

(51) Int. Cl. A61B 5/11; A61B 5/00

(52) U.S. Cl. CPC A61B 5/1114; A61B 5/7257; A61B 5/7267; A61B 5/1128

(57) ABSTRACT

A method and system for the detection of dyslexia utilizing machine learning Discrete Fourier Transformation technique in conjunction with Support Vector Machine on eye tracking data. Eye movements of a user engaged in reading are captured and processed to generate a dataset. This dataset is then transformed from the time-domain into the frequency-domain using a discrete Fourier transformation technique. The frequency-domain representation is subsequently input into a Support Vector Machine with a linear kernel trained to identify patterns indicative of dyslexia. The system outputs a result based on this analysis, indicating the potential presence or absence of dyslexic tendencies in the user.

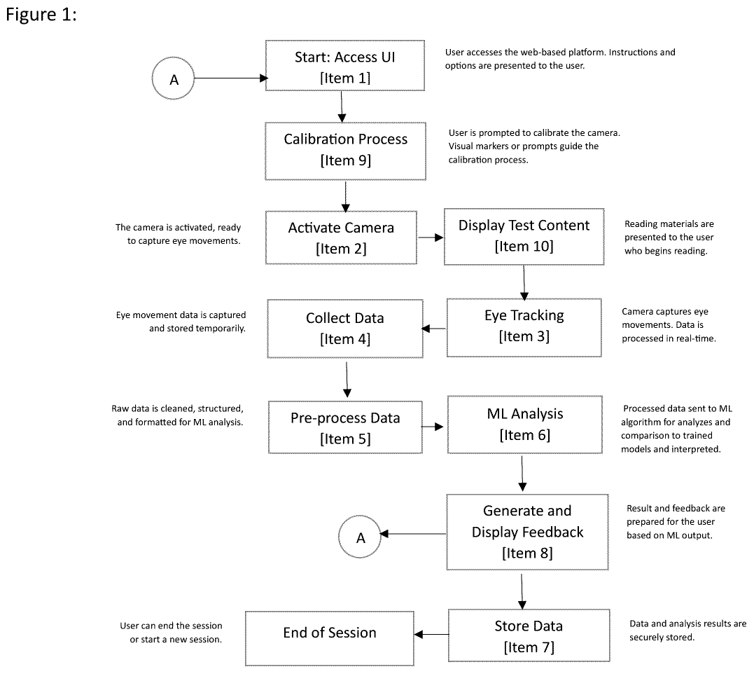

### CROSS-REFERENCE TO RELATED APPLICATIONS

### Federally Sponsored Research and Development Joint Research Agreement

### Reference to a “Sequence Listing”, a Table, or a Computer Program Listing Appendix Submitted on a Compact Disc and an Incorporation-by-Reference of the Material on the Compact Disc

### Prior Art

### Background of the Invention

### Purpose of the Invention:

What problem does the invention solve?

The invention addresses the challenge of early and efficient detection of dyslexia in individuals. Traditional methods of dyslexia identification can be time-consuming, require specialized personnel, and may not always be accessible to everyone. Furthermore, early intervention is crucial for individuals with dyslexia, yet many are not diagnosed until they face significant academic challenges.

How does the invention solve the problem described above?

The invention utilizes a machine learning algorithm hosted on a website, allowing users to easily access and engage with the tool from anywhere. By analyzing the user's eye movements, the algorithm can swiftly and accurately identify patterns consistent with dyslexia. This digital approach eliminates the need for specialized personnel for initial screening, making the detection process more efficient and widely accessible. As a result, individuals can receive a prompt preliminary assessment, facilitating earlier interventions and support.

How is the invention an Improvement? How is the invention different from and better than anything that exists in its field?

Previous machine learning models for dyslexia detection had focused only on the number of jumps or fixations in a user's eye movement; however, this invention uses Support Vector Machines and the Discrete Fourier Transform to break down patterns in the eye-movement data, taking into context how long or frequent the jumps and fixations are. Furthermore, by being hosted online, it democratizes access to dyslexia screening, ensuring that a broader audience can benefit from early detection and intervention.

How is the invention an Improvement? (continued)

What are the problems with the other devices or systems in the field of the invention?

Existing systems in the field predominantly rely on manual cognitive and linguistic assessments, which can be lengthy and might not capture the subtle visual processing differences associated with dyslexia. They often rely on lengthy, manual assessments that necessitate specialized personnel, making them less accessible and often leading to delayed diagnoses. Additionally, traditional methods may not always adapt to the evolving understanding of dyslexia, potentially missing subtle indicators or nuances in individual cases.

Why don't these devices or systems work well?

These traditional systems are constrained by their reliance on manual evaluations, which can be time-consuming and subject to human error or oversight. Their limited accessibility and adaptability mean that many individuals might not receive timely or comprehensive assessments, hindering early intervention efforts. Traditional methods, while effective in many cases, might miss individuals whose primary dyslexic indicators are visual rather than linguistic. They also require specialized personnel and can be time-consuming, leading to potential delays in diagnosis and intervention.

How does this invention improve on them?

My invention harnesses eye movement and tracking data, offering a unique lens into the visual processing aspects of dyslexia. By integrating machine learning algorithms, specifically Support Vector Machines, in conjunction with the Discrete Fourier Transform, the system can analyze this data with high precision, providing a more comprehensive and nuanced assessment than traditional methods. Being web-based, it also ensures broader accessibility and faster results.

### SUMMARY OF THE INVENTION

Items or Steps that Make Up The Invention
List of the individual components or elements that make up the invention. Each item is numbered according to the accompanying drawing.
1. User Interface (UI): A web-based platform where users can interact with the application.
2. Camera to track the user's eye movement: A camera to capture detailed eye movements in real-time.
3. Eye tracking module: A software component that tracks the eye movement of the user.
4. Data Collection Module: A software component that collects and stores the raw data from the eye-tracking component.
5. Pre-processing Module: This module cleans and structures the raw eye-tracking data, making it suitable for analysis.
6. Machine Learning Algorithm: The core analytical component that has been trained on previous datasets to recognize patterns consistent with dyslexia.
7. Files: A storage system where user data, eye movement patterns, and analysis results are stored securely.
8. Feedback Module: After analysis, this module provides users with results, either confirming typical reading patterns or suggesting potential dyslexic tendencies.
9. Calibration Module: Before the test, this module ensures the eye-tracking camera is correctly calibrated to the user's position and eye movements.
10. Test Content Display: A set of reading materials or visual stimuli presented to the user during the test to induce eye movements for analysis.

### BRIEF DESCRIPTION OF DRAWINGS

Relationship Between the Components

Description of the relationship between the invention's components, elements and steps, using the same item numbers as in the accompanying drawing.
1. User Interface (UI) serves as the primary point of interaction for users. It integrates all other components, offering a cohesive experience from start to finish.
2. When a user starts the test via the UI, the camera begins capturing the intricate movements of the eyes.
3. Eye tracking module interfaces directly with the camera (2). As the camera captures the eye movements, this module actively tracks and maps these movements in real-time.
4. Data Collection Module is in sync with the Eye tracking module (3). It gathers the tracked eye movement data and temporarily stores this raw information for further processing.
5. The Pre-processing Module takes over once the raw data is collected. It receives data from the Data Collection Module (4), refining it by removing noise and ensuring it's in a format ready for analysis.
6. Using Support Vector Machines and Discrete Fourier Transformation, processes the cleaned data from the Pre-processing Module (5). It analyzes the data, comparing it with previously trained datasets to identify patterns indicative of dyslexia.
7. Files serve as the primary storage. They interact with multiple components: the Data Collection Module (4) saves raw data here, the Machine Learning Algorithm (6) might retrieve past datasets for training, and the Feedback Module (8) could access past results for comparative feedback.
8. Feedback Module interprets the results from the Machine Learning Algorithm (6) and conveys them to the user via the UI (1), offering insights and potential diagnoses based on the analyzed eye movements.
9. Calibration Module is a preparatory step accessible via the UI (1). Before any test begins, it ensures the Camera (2) is accurately capturing data by calibrating it to the user's specific position and eye movements.
10. Test Content Display is an integrated part of the UI (1). Post-calibration, the user is presented with reading materials or visual stimuli. These materials are designed to induce specific eye movements, which the Camera (2) captures and the Eye tracking module (3) tracks.

Relationship Between the Components (continued)

Description of the logic required for creation, implementation, and functioning of the invention.
1. Initialization Logic: Upon accessing the UI (1), the system initializes by verifying the readiness of the Camera to track the user's eye movement (2) and the Calibration Module (9). If the camera isn't detected or functional, an error message is displayed.
2. Calibration Logic: Post initialization, the Calibration Module (9) activates. Using “if-then” gates, if the calibration is successful, the user proceeds to the test. If calibration fails, the user is prompted to recalibrate or check their camera setup.
3. Test Logic: On successful calibration, the Test Content Display (10) showcases reading materials or visual stimuli. Concurrently, the Camera (2) begins capturing eye movements, and the Eye tracking module (3) initiates its tracking subroutine.
4. Data Collection Logic: As the eye movements are tracked, the Data Collection Module (4) continuously gathers this data. Using a conditional gate, if the test concludes or the user halts the test, data collection ceases, and the data is relayed to the Pre-processing Module (5).
5. Processing Logic: The Pre-processing Module (5) refines the data, and activates the ML Algorithm (6) which uses discrete Fourier transformation and a Support Vector Machine to analyze the data. If patterns consistent with dyslexia are identified, a flag is set; otherwise, a different flag indicating typical reading patterns is set.
6. Storage Logic: After the analysis, the raw and processed data, with the results, are archived in Files (7).
7. Feedback Logic: Depending on the flag determined by the ML Algorithm (6), the Feedback Module (8) retrieves the appropriate feedback. Using an “if-then-else” gate, if the dyslexia flag is activated, feedback suggesting potential dyslexic tendencies is presented; otherwise, feedback confirming typical reading patterns is shown.
8. Loop Logic: Post-feedback, users are offered an option to retake the test or exit. If they opt to retake, the system loops back to the Calibration Logic (2); if not, the session concludes.

### DETAILED DESCRIPTION OF THE INVENTION

How Does The Invention Work?

How do the components, steps or elements of the invention work individually and together to cause the whole invention to perform its desired function?
1. User Interface (UI): This is the front-end component where users initiate and interact with the entire system. It provides a user-friendly environment, guiding users through calibration, testing, and feedback stages.
2. Camera to track the user's eye movement: This hardware component captures the intricate movements of the user's eyes in real-time. It's the primary data collection point, capturing visual cues that are crucial for the detection process.
3. Eye tracking module: Working in tandem with the camera, this software component processes the visual data in real-time, mapping and tracking the movement and focus points of the eyes as the user engages with the content.
4. Data Collection Module: This module acts as a bridge, collecting the tracked eye movement data from the Eye tracking module and storing it temporarily for further processing. It ensures that all relevant data points are captured and ready for the next steps.
5. Pre-processing Module: Before the data can be analyzed, it needs to be refined. This module cleans the raw eye-tracking data, removing any noise or irrelevant information, and structures it in a format suitable for the Machine Learning Algorithm.
6. Machine Learning Algorithm: Using discrete Fourier transform, the algorithm breaks down the clean data into a sum of individual frequencies along with their corresponding amplitudes. Selecting the frequencies which correspond to the second to fifth highest amplitudes, the algorithm then uses a pre-trained Support Vector Machine with a linear kernel to classify the user as dyslexic or non-dyslexic based on these frequencies.
7. Files: This is the secure storage system. All raw and processed data, along with the results of the analysis, are stored here. It ensures that data integrity is maintained and that the system can retrieve past results if needed for comparative analysis.
8. Feedback Module: Once the Machine Learning Algorithm completes its analysis, this module translates the results into understandable feedback for the user. Depending on the analysis, it provides insights, either confirming typical reading patterns or suggesting potential dyslexic tendencies.
9. Calibration Module: Before any testing begins, it's crucial that the eye-tracking camera accurately captures data. This module ensures the camera is correctly calibrated to the user's position and eye movements, ensuring accuracy in the data collection phase.
10. Test Content Display: This is where the user interacts with reading materials or visual stimuli. Designed to induce specific eye movements, the content here is crucial for the detection process. As the user engages with this content, the camera and Eye tracking module work in tandem to capture all relevant data.
In Summary: The invention operates as a cohesive system. The user interacts with the UI, going through calibration and then engaging with test content. As they do so, their eye movements are captured, tracked, and stored. This data is then cleaned, processed, and analyzed by a sophisticated machine learning algorithm. Finally, the user receives feedback based on this analysis, all in a streamlined and user-friendly manner.

How to Make the Invention:

How would a person make the invention?

1. Setting up the User Interface (UI):
Choose a web development framework suitable for interactive applications.
Design a user-friendly layout with clear instructions, buttons for calibration, testing, and viewing feedback.
Ensure the UI is responsive, making it accessible on various devices, from desktops to mobiles.

2. Integrating the Camera for Eye Movement:
Procure a high-resolution camera capable of capturing detailed eye movements in real-time.
Integrate the camera's API with the web platform, ensuring it can be activated and controlled via the UI.

3. Developing the Eye Tracking Module:
Use computer vision libraries, to process the visual data from the camera.
Implement algorithms to detect, map, and track the movement and focus points of the eyes as the user engages with content.

4. Creating the Data Collection Module:
Develop a module that can capture the tracked eye movement data in real-time.
Store this data temporarily in a buffer or cache for immediate processing.

5. Designing the Pre-processing Module:
Implement algorithms to clean the raw eye-tracking data, removing noise or irrelevant information.
Structure the data in a format suitable for machine learning analysis, such as arrays or matrices.

6. Implementing the Machine Learning Algorithm:
Choose a machine learning model, such as Support Vector Machines.
Train a model using datasets of eye movements, both typical and those consistent with dyslexia.
Integrate discrete Fourier transformation techniques to analyze the cleaned data and compare it against the trained model.

7. Setting up Files for Storage:
Choose a secure file system to efficiently store raw data, processed data, and analysis results.

8. Developing the Feedback Module:
Create algorithms to interpret the results from the machine learning analysis.
Design feedback templates in the UI to display insights to the user in an understandable manner.

9. Building the Calibration Module:
Develop a subroutine where users can calibrate the camera before testing.
Use visual markers or prompts on the UI to guide users through the calibration process, ensuring the camera accurately captures their eye movements.

10. Designing the Test Content Display:
Curate or design reading materials or visual stimuli known to induce specific eye movements.
Integrate these materials into the UI, ensuring they are displayed clearly and are easily readable.

In Summary: To make the invention, one would need a combination of hardware (a high-resolution camera) and software components (web platform, computer vision libraries, machine learning frameworks). The process involves setting up a user-friendly interface, integrating real-time eye tracking, processing and analyzing the captured data, and providing feedback to the user. Proper calibration and test content are crucial for accurate results. Throughout the development, ensure that user data is stored securely, and that the system operates seamlessly from start to finish.

Which elements are necessary?
The User Interface (UI) is the primary interaction point for users.
Camera Integration enables real-time eye tracking, central to the invention.
The Eye Tracking Module processes visual data, tracking eye movements.
Data Collection captures and stores eye movement data.
The Machine Learning Algorithm analyzes data to identify dyslexia patterns.
The Feedback Module provides users with analysis insights.

Which elements are optional?
The Pre-processing Module refines data but could be integrated elsewhere.
The Calibration Module is optional with advanced adaptive systems.
Test Content Display is optional if users provide their own materials.

What elements could enhance the invention?
User Profiles for personalized feedback and progress tracking.
Adaptive Learning for tailored reading materials.
Real-time Feedback for immediate user insights.
AR Integration: AR glasses might offer natural reading scenarios.
Cloud Integration for easier data access and sharing.

How to Use the Invention:

How would a person use the invention to solve the problem that the invention solves?
1. Access the Platform:
   Users start by navigating to the web-based User Interface (UI) using a compatible device with a camera, preferably a computer or tablet for optimal screen size.
2. Calibration:
   If the system has a Calibration Module, users should follow on-screen instructions to calibrate the camera. This ensures accurate tracking of eye movements. They might be guided by visual markers or prompts on the UI.
3. Choose Reading Material:
   If the system doesn't provide specific content, users should open their reading material on the Test Content Display. If the system provides content, users select from available options.
4. Begin Eye Tracking Session:
   Users initiate the eye tracking session via a button or command on the UI.
   As they read the content, the Camera captures their eye movements, which are then processed by the Eye Tracking Module.
5. Data Collection and Analysis:
   The Data Collection Module gathers the tracked data in real-time.
   This data is then either pre-processed or directly fed into the Machine Learning Algorithm. The algorithm analyzes the data, looking for patterns indicative of dyslexia.
6. Receive Feedback:
   Once the analysis is complete, the Feedback Module presents users with results on the UI.
   Feedback might confirm typical reading patterns or suggest potential dyslexic tendencies based on the analysis.
7. Review and Action:
   Users can review the feedback, understanding their reading patterns and any potential signs of dyslexia.
   If dyslexic tendencies are indicated, users might be advised to seek further professional assessment or provided with resources to assist with reading challenges.
In Summary: To use the invention, a person accesses the platform, calibrates the system, selects or provides reading material, and initiates an eye tracking session. As they read, their eye movements are captured, analyzed, and then feedback is provided. This feedback helps users understand their reading patterns and any potential dyslexic tendencies.

Can this invention be used in a different way or in another field of technology?
YES

Other Invention Uses:

Describe how this invention can be used in a different way or in another field of technology.
1. Educational Tools:
   The invention can be integrated into e-learning platforms to monitor student engagement. By tracking eye movements, educators can determine which parts of the content are most engaging or where students might be struggling.
2. Usability Testing:
   In the field of web and software design, the invention can be used to conduct usability tests. Designers can understand where users focus most, helping refine user interfaces for better user experience.
3. Marketing and Advertising:
   Companies can use the invention to test the effectiveness of their advertisements or website designs. By analyzing where viewers' eyes are drawn, marketers can optimize ad placements or design elements for maximum impact.
4. Gaming:
   Game developers can integrate the invention to enhance player immersion. For instance, in virtual reality (VR) or augmented reality (AR) games, characters or elements could respond to where players are looking.
5. Healthcare:
   Beyond dyslexia, the invention can be adapted to detect other neurological or cognitive conditions that might manifest through eye movements, such as certain types of attention disorders or even early signs of neurodegenerative diseases.
6. Security Systems:
   The invention can be incorporated into security systems for biometric identification based on unique eye movement patterns or for lie detection during interrogations.
7. Automotive Industry:
   In modern vehicles, the invention can be used to monitor driver alertness. If the system detects patterns consistent with drowsiness or distraction, it can alert the driver or even take corrective actions.
8. Research:
   In academic or industrial research settings, the invention can be used to study human behavior, cognition, or reactions to various stimuli, providing valuable data for a range of studies.
Besides dyslexia detection, the invention's eye-tracking can be used in education, marketing, gaming, and healthcare.

Can the invention produce a product, device, composition, or other useful item?
YES

Other Invention Uses (continued)

Please list and describe all products and devices, compositions and other useful items that your invention can produce.
1. Engagement Metrics Tool:
   A software product that provides educators or content creators with metrics on user engagement, highlighting areas of interest or difficulty based on eye movement patterns.
2. User Experience (UX) Optimizer:
   A tool for web and app developers to refine interfaces. It provides heatmaps and focus charts based on where users predominantly look or get stuck.
3. Ad Efficacy Analyzer:
   A product for marketers to gauge the effectiveness of digital advertisements by tracking viewer attention and dwell time on specific ad elements.
4. Interactive Gaming Enhancer:
   A software module for game developers, allowing in-game elements to react to player gaze, creating a more immersive experience.
5. Neurological Diagnostic Aid:
   A medical tool that tracks eye movements to assist in diagnosing other neurological conditions, potentially serving as an early detection system.
6. Biometric Security Software:
   A security product that uses unique eye movement patterns for identity verification, adding an extra layer of biometric security.
7. Driver Alertness Monitor:
   A device for vehicles that monitors driver eye movements, sounding alarms or notifications if patterns suggest drowsiness or distraction.
8. Behavioral Research Tool:
   A software suite for researchers studying human cognition, behavior, or reactions, providing detailed eye-tracking data for analysis.
The invention's core technology can be the foundation for various products and tools across multiple sectors, enhancing user experience, security, research, and healthcare diagnostics.

1. A method for detecting dyslexia using eye tracking data, comprising the steps of:
a) capturing eye movement data of a user while engaged in a reading activity using an eye tracking system;
b) processing said eye movement data to generate a formatted dataset suitable for analysis;
c) applying a discrete Fourier transformation technique to said formatted dataset to convert the time-domain eye movement data into a frequency-domain representation;
d) inputting said frequency-domain representation into a Support Vector Machine trained to recognize patterns consistent with dyslexia; and
e) outputting a result based on the analysis of said machine learning model, wherein said result indicates the presence or absence of dyslexic tendencies in the user.
